(19) United States
(12) Patent Application Publication
(10) Pub. No.: US 2026/0043789 A1
(43) Pub. Date: Feb. 12, 2026

(54) DISPOSABLE CHEMICAL SENSOR ARRAYS AND BREATH MONITORING SYSTEM

(71) Applicant: YAZAKI CORPORATION, Tokyo (JP)

(72) Inventors: Zhexiong Tang, Camarillo, CA (US); Stefan Maat, Camarillo, CA (US)

(21) Appl. No.: 19/365,097

(22) Filed: Oct. 21, 2025

Related U.S. Application Data
(63) Continuation of application No. 18/161,854, filed on Jan. 30, 2023.

(51) Int. Cl. G01N 33/497

(52) U.S. Cl. CPC G01N 33/497; G01N 33/4975

(57) ABSTRACT

A device for detecting one or more gases of ammonia (NH₃), trimethylamine (TMA), and hydrogen sulfide (H₂S) includes a substrate and an electrodes layer. The device also includes a gas sensor array including one or more gas sensing films, each electronically coupled with an electrode. Each gas sensing film is configured to chemically interact with a respective target gas. Each electrode is configured to measure resistance changes across the respective gas sensing film to which it is electronically coupled. The multiple gas sensing films include one or more of polyaniline (PANI) doped with camphor sulfonic acid (CSA) for chemically interacting with NH₃ and/or TMA, PANI doped with 4-dodecylbenzenesulfonic acid (DBSA) for chemically interacting with TMA and/or NH₃, and metal salt-doped PANI with poly(3,4-ethylenedioxythiophene) polystyrene sulfonate (PEDOT:PSS) composite for chemically interacting with H₂S.

### CROSS-REFERENCE TO RELATED APPLICATIONS

This application is a continuation of U.S. patent application Ser. No. 18/161,854, filed Jan. 30, 2023, incorporated herein by reference in its entirety.

### TECHNICAL FIELD

The disclosed teachings relate to chemical sensor devices and chemical sensor systems. In particular, the disclosed teachings relate to disposable chemical sensor arrays for detection of one or more gases and breath analyzer systems that can implement detection of the one or more gases using the disposable chemical sensor arrays.

### BACKGROUND

Human breath is a complex environment with high relative humidity (e.g., relative humidity above 90%) and with several hundreds of volatile organic compounds (VOCs). Their concentrations range from a few parts per trillion (ppt) up to thousands of parts per million (ppm) within human breath samples. Moreover, there is great variability of the VOC composition between individuals which depends on many factors such as age, gender, diet, and health, among others. Over the last decade, correlations between VOCs present in the breath and diseases of the gastrointestinal tract, the kidneys, the lungs, the liver, metabolic disorders, and cancer have been studied. Specific VOCs are considered to be biomarkers of such diseases and conditions.

Currently, VOCs are detected using technologies such as gas chromatography-mass spectroscopy (GC-MS), selective ion flow tube mass spectrometry (SIFT-MS), and field asymmetric ion mobility spectrometry (FAIMS). However, none of these instruments are small, hand-held, inexpensive, and available for use over-the-counter (OTC). Neither, are any of these instruments capable of analyzing human exhalation directly through an opening of the instrument or through a mouthpiece. Also, current commercially available breath analyzers are typically designed to detect only a single analyte such as an alcohol detector.

Furthermore, due to the cross-sensitivity of individual sensor materials toward close analytes such as ammonia (NH₃) and trimethylamine (TMA) in breath samples, it is challenging to use a single sensing material to detect these analytes selectively based on sensor resistance change. As such, there remains a need for a reliable point-of-care diagnostic test that can be self-administered using a hand-held device to unambiguously detect VOCs or other compounds in breath samples.

### BRIEF DESCRIPTION OF THE DRAWINGS

One or more embodiments of the present disclosure are illustrated by way of example and not limitation in the Figures of the accompanying drawings, in which like references indicate similar elements.

FIG. 1A-1C are schematic illustrations of a chemical gas sensor device and chemical gas sensor arrays in accordance with some embodiments.

FIGS. 2A-2B are schematic illustrations of a sensor electrode and a sensor inductive coil of the chemical gas sensor device of FIG. 1A in accordance with some embodiments.

FIG. 3A-3B are schematic illustrations of disposable chemical gas sensing strips in accordance with some embodiments.

FIG. 4 is a schematic illustration of a breath analyzer device in accordance with some embodiments.

FIG. 5A is a schematic illustration of a breath analyzer system in accordance with some embodiments.

FIG. 5B is a schematic illustration of an electronic connection port of the breath analyzer system of FIG. 5A in accordance with some embodiments.

FIG. 5C is a schematic illustration of an exemplary environment where the breath analyzer system of FIG. 5A can operate in accordance with some embodiments.

FIG. 6 illustrates a chemical mechanism for PANI-based detection of NH₃.

FIG. 7 is a schematic illustration of the fabrication route of emeraldine salt of PANI doped with CSA.

FIG. 8A illustrates a bare PET substrate with PANI_CSA_{0.75} thin films prepared using a dip coating apparatus.

FIG. 8B illustrates a PANI_CSA_{0.75} thin film dip coated onto a bare PET substrate.

FIG. 9 illustrates an optical microscope image of the prepared interdigitated electrode layer.

FIG. 10 illustrates the fabrication process of chemical gas sensors including PANI-based conducting polymers doped with CSA in accordance with some embodiments.

FIG. 11 illustrates the fabrication process of PANI-based conducting polymers doped with CSA in accordance with some embodiments.

FIG. 12 is a schematic illustration of the fabrication route of emeraldine salt of PANI (PANI(ES)) doped with DBSA.

FIG. 13 illustrates a sensing mechanism of Sn/Cu (II) doped PANI composites for H₂S.

FIG. 14 is a schematic illustration of formulation routes for Sn/Cu (II) doped PANI-PEDOT:PSS composite solutions for spray coating or dip coating.

FIGS. 15, 16A and 16B are graphical illustrations of resistance changes of a CSA-doped PANI sensor device when exposed to NH₃ gas.

FIG. 17 is a graphical illustration of resistance changes of PANI_DBSA_{1.5} sensors when exposed to TMA gas.

FIGS. 18A and 18B are graphical illustrations of resistance changes of Sn(II) doped PANI-PEDOT:PSS composites when exposed to H₂S under 90% RH.

FIG. 19 is a graphical illustration of sensor array response patterns for three different chemical gas sensors.

FIG. 20 is a graphical illustration of classification of H₂S, TMA, NH₃, and TMA-NH₃ based on principle component analysis (PCA).

FIG. 21 is a schematic illustration of an inductive coil.

FIGS. 22A and 22B are graphical illustrations of resonance impedance spectrum changes of PANI_CSA_{0.5} coated inductive coils for TMA-NH₃ concentration monitoring.

### DETAILED DESCRIPTION

### Abbreviations

CSA: camphor sulfonic acid
DBSA: 4-dodecylbenzenesulfonic acid
H₂S: hydrogen sulfide
NH₃: ammonia
m-cresol: 3-methylphenol
PANI: polyaniline
PEDOT:PSS: poly(3,4-ethylenedioxythiophene): polystyrene sulfonate
TMA: trimethylamine

### Chemical Structures

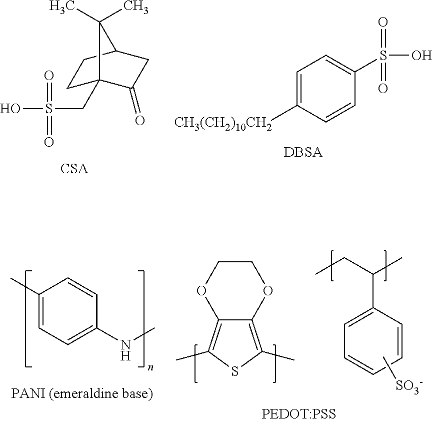

The present invention provides a chemical gas sensor array containing multiple sensor elements to overcome selectivity issues during breath analysis.

The present technology is directed to gas sensing devices and breath analyzer systems that are configured for the detection of multiple chemical gases. The gas sensing device includes the chemical gas sensor array which is comprised of a substrate, one or more sensing electrodes, and multiple gas sensing films. The gas sensing films can include films based on the conductive polymer polyaniline (PANI). The multiple PANI-based gas sensing films can include different dopants and/or additives and be configured to detect different gases. For example, the breath analyzer systems including the gas sensing device can be configured to detect and differentiate between hydrogen sulfide, ammonia, and trimethylamine in human breath samples within a few seconds of sampling time.

### Chemical Sensor Device

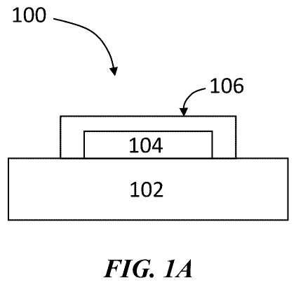

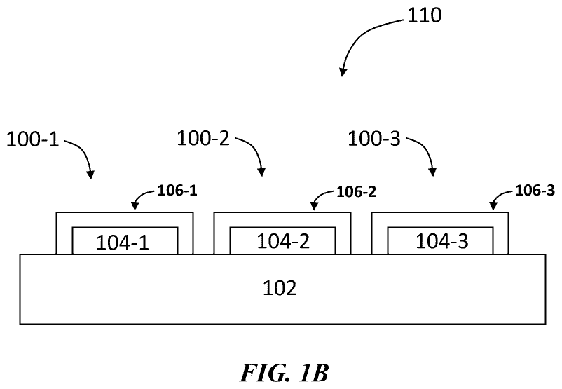

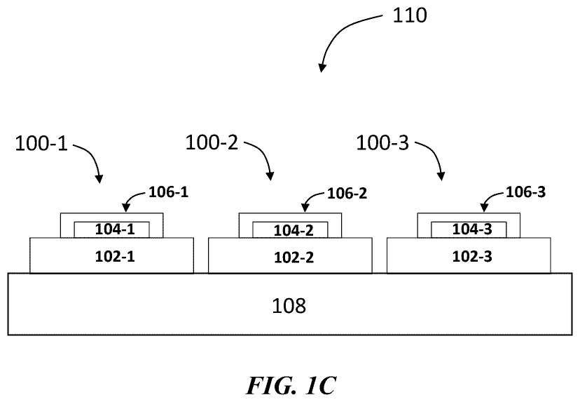

FIG. 1A is a schematic illustration of a chemical gas sensor device 100 in accordance with some embodiments. The chemical gas sensor device 100 is for detecting one or more gases of ammonia, trimethylamine, and hydrogen sulfide. The device includes a substrate 102, an electrode 104, and a gas sensing film 106 configured in a stack, as shown. The gas sensing film may contact the substrate 102, as shown, or it may contact only the electrode 104.

FIG. 1B is a schematic illustration of a chemical gas sensor array 110 in accordance with some embodiments. The chemical gas sensor array 110 is for detecting and differentiating among multiple chemical gases including, for example, ammonia, trimethylamine, and hydrogen sulfide. The array includes multiple chemical gas sensor devices 100-1, 100-2, 100-3, etc. Each chemical gas sensor device is configured in a stack that includes a respective electrode 104-1, 104-2, 104-3, etc., and a respective gas sensing film 106-1, 106-2, 106-3, etc. All the electrodes and their respective gas sensing films are supported on a single substrate 102.

FIG. 1C is a schematic illustration of a variation of chemical gas sensor array 110 in accordance with some embodiments. In this case, Each chemical gas sensor device 100-1, 100-1, and 100-3 has its own respective, discrete substrate 102,1, 102-3, and 102-3. These substrates may or may not, as shown, be in contact with one another. All the chemical gas sensing devices 100-1, 100-2, and 100-3 are supported on a base 108.

The chemical gas sensor array 110 can include one or more chemical gas sensing devices. In some embodiments, the chemical gas sensor array 110 includes multiple chemical gas sensing devices 100-1, 100-2, 100-3, etc. Each chemical gas sensing device within the array includes a gas sensing film (e.g., gas sensing films 106-1, 106-2, and 106-3) that is each electronically connected to a respective sensor electrode 104-1, 104-2, and 104-3. In some embodiments, the chemical gas sensor array 110 includes more than three chemical gas sensing devices (e.g., four, five, six, seven, eight, nine, ten, or more devices). Each of the gas sensing films 106-1, 106-2, 106-3, etc. in respective devices 100-1, 100-2, 100-3, etc. is configured to chemically interact with one or more target gases. For example, the target gases can be selected from NH₃, TMA, and H₂S.

Each of the gas sensing films 106-1, 106-2, and 106-3 can include, for example, PANI-based conducting polymer doped with CSA, PANI-based conducting polymer doped with DBSA, or metal salt (e.g., Sn²⁺ and/or Cu²⁺) doped PANI with PEDOT:PSS composite. The PANI-based conducting polymer doped with CSA is configured to chemically interact with NH₃ and/or TMA, the PANI-based conducting polymer doped with DBSA is configured to chemically interact with TMA and/or NH₃, and the metal salt-doped PANI with PEDOT:PSS composite is configured to chemically interact with H₂S. As used herein, a chemical interaction may refer to any type of interaction between the gas sensing film and a target chemical that is detectable by the chemical sensor device 100. The chemical interaction can include, for example, covalent bonding, non-covalent bonding, or ionic interaction.

In some embodiments, the thickness of the respective gas sensing film is less than or equal to 100 nm (e.g., the gas sensing film is less than or equal to 100 nm, less than or equal to 80 nm, less than or equal to 60 nm, less than or equal to 50 nm, less than or equal to 40 nm, or less than or equal to 30 nm). In some embodiments, the gas sensing film has a thickness that is about 50 nm or about 30 nm. The film thickness of the gas sensing films is significant for sensitivity and response time for gas sensing in breath samples. In some embodiments, the device is configured to have a detection limit of 0.1 ppm to 0.5 ppm for at least one of NH₃, TMA, and H₂S. For example, nano-scale (<50 nm) ultrathin films have demonstrated a gas sensing performance under simulated breath conditions (>90% relative humidity (RH) air mixtures) with a detection limit of 0.1 to 0.5 ppm for at least one or more of NH₃, TMA, and H₂S with the detection time of 15 seconds (e.g., as described in Examples 5-7).

As described above, chemical gas sensor array 110 is comprised of a plurality of chemical gas sensing devices (100-1, 100-2, 100-3, etc.), each of which is comprised of a gas sensing film (106-1, 106-2, 106-3, etc.) Each gas sensing film may consist of any one of a PANI-based film doped with CSA, a PANI-based film doped with DBSA, or a PANI-PEDOT:PSS hybrid material doped with a metal salt.

In some embodiments, a molar ratio of CSA to PANI is in a range of 1 to 1.5 (e.g., the molar ratio of CSA to PANI is ranging from 1 to 1.25, from 1.25 to 1.5, or from 1.2 to 1.5). In some embodiments, a molar ratio of DBSA to PANI is in a range of 1 to 1.5 (e.g., the molar ratio of DBSA to PANI is ranging from 1 to 1.25, from 1.25 to 1.5, or from 1.2 to 1.5). In some embodiments, a weight percentage of PEDOT:PSS in the metal salt-doped PANI with PEDOT:PSS composite is in a range of 1 wt % to 4 wt % (e.g., in the range of 1 wt % to 2 wt %, 2 wt % to 3 wt %, or 3 wt % to 4 wt %). In some embodiments, the weight percentage of PEDOT:PSS in the metal salt doped PANI with PEDOT:PSS composite is about 3 wt %.

As shown in FIG. 1A, the sensor electrodes 104 can be positioned between the substrate 102 and the gas sensing film 106. Alternatively, the gas sensing film 106 can be positioned between the substrate 102 and the sensor electrodes 104.

The gas sensing film 106 may cover the top and side surfaces of the sensor electrodes 104 such that it is in contact also with the substrate 102, as shown in FIG. 1A. Or, the gas sensing film 106 may be in contact only with the sensor electrodes 104 and not be in contact with the substrate 102.

In some embodiments, the chemical gas sensor device 100 includes two gas sensing films 106. For example, the chemical gas sensor device 100 includes a first gas sensing film that is positioned on a first surface of the substrate 102 and a second gas sensing film that is positioned on a second surface of the substrate 102. The second surface is opposite to and parallel with the first surface of the substrate. The sensor electrodes 104 can be positioned either between the substrate 102 and either of the first or second gas sensing films or either the first gas sensing film or the second gas sensing film is positioned between the substrate 102 and the sensor electrode 104. The sensor electrodes 104 are electronically connected with the respective adjacent gas sensing film.

In some embodiments, the gas sensing films 106-1, 106-2, and 106-3 are prepared by forming a PANI-based sensor layer onto one or two surfaces of the substrate 102 by printing and/or coating methods known in the art, including, for example, gravure printing, screen printing, flexographic printing, ink-jet printing, spin coating, spray coating, dip coating, and roll-to-roll coating techniques such as slot-die, comma, and reverse comma coating (see Example 1 which includes sensor fabrication processes). The printing and/or coating of the PANI based sensor layer is followed by a curing process (e.g., ultraviolet (UV), heat, or chemical curing). In instances where the sensor electrode 104 is positioned between the substrate 102 and the gas sensor array 106, the gas sensing films 106-1, 106-2, and 106-3 are prepared on a surface of the sensor electrode 104 instead of on the substrate 102.

In some embodiments, the substrate 102 is made of an insulating material, such as ceramic, polymer, paper, cardboard, or any other suitable insulating material. The polymer can include, for example, polyethylene terephthalate (PET), polycarbonates (PC), polyamides (PA), polyimides (PI), poly(methyl methacrylate) (PMMA), or any other suitable polymer material. In some embodiments, the substrate 102 is flexible (e.g., non-rigid).

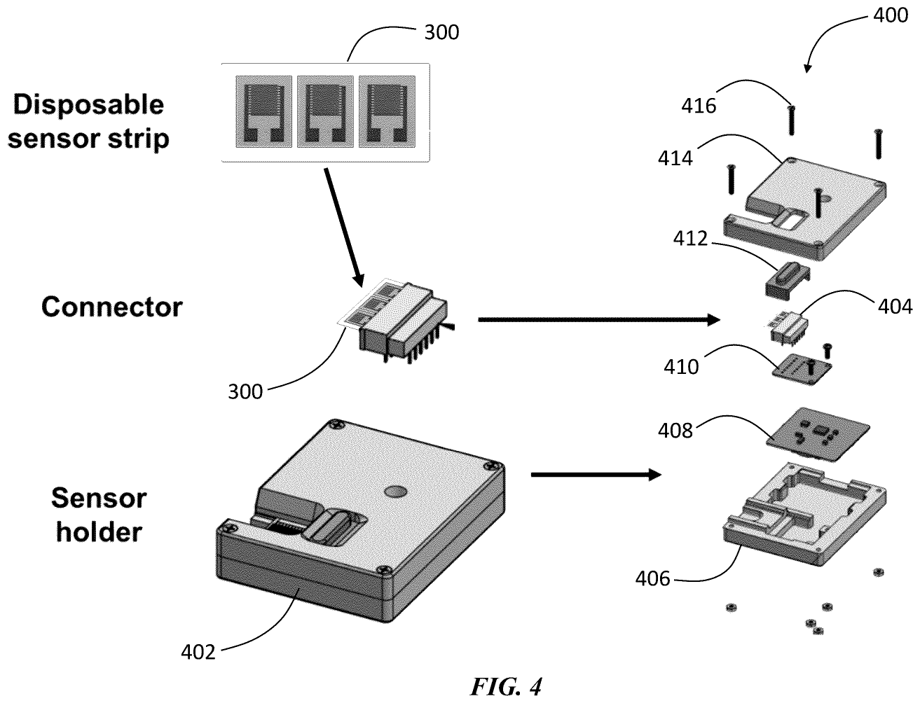

In some embodiments, the chemical sensor device 100 is configured as a disposable chemical sensor device. As used herein, disposable refers to a device that is made of low-cost materials and that is configured to be disposed of after usage. For example, the substrate 102, the sensor electrodes 104, and the gas sensor array 106 are made of low-cost materials that can be used for chemical sensing while inserting the chemical sensor device 100 to a chemical sensor system (e.g., as described in FIG. 4A) and disposed of after being used. The sensor electrodes 104 are configured to measure resistance and/or capacitance across each of the gas sensing films 106-1, 106-2, and 106-3 upon chemical interaction of the gas sensing film with a target gas.

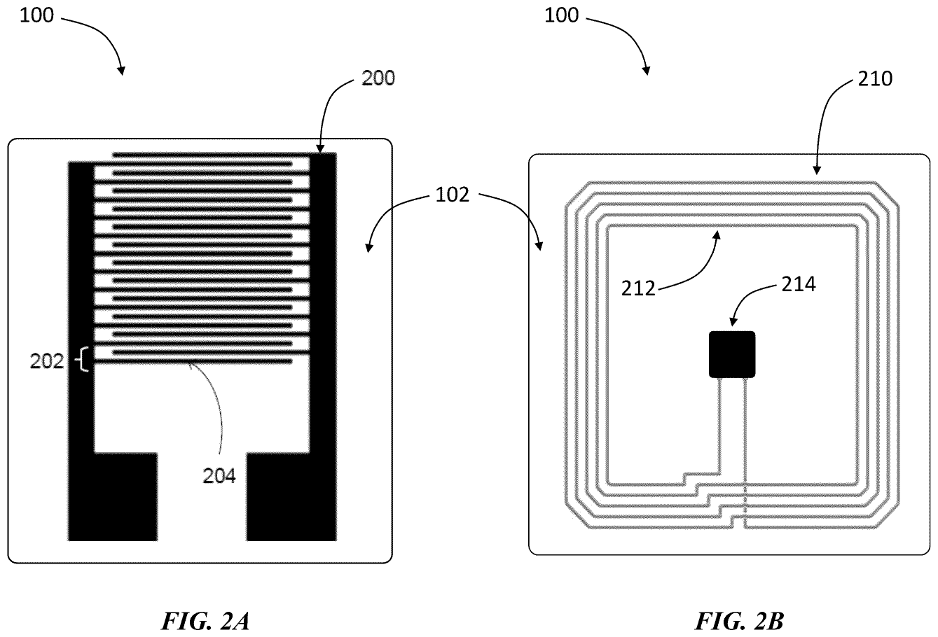

FIGS. 2A-2B are schematic illustrations of chemical sensor devices 100 of FIG. 1A in accordance with some embodiments.

The interdigitated electrode layer 200 according to one embodiment and shown in FIG. 2A is deposited onto substrate 102. The interdigitated electrode layer 200 includes multiple pairs of electrodes 202 (e.g., six or twelve electrode pairs). Each gas sensing film 106-1, 106-2, and 106-3 of gas sensor array 110 is in contact with a separate and discrete interdigitated electrode layer 200.

In some embodiments, a width of an electrode line (e.g., a width of an electrode line 204 measured in a vertical direction in FIG. 2A) in the interdigitated electrode layer 200 is between 100 μm and 1000 μm and a spacing between respective electrode lines (e.g., a spacing between the electrode lines 204 measured in a vertical direction) is between 100 μm and 1000 μm. For example, the width of the electrode line 202 can be between 100 μm and 1000 μm, between 100 μm and 500 μm, between 500 μm and 1000 μm, between 200 μm and 500 μm, or between 200 μm and 300 μm. For example, the width of the spacing between respective electrode lines 204 can be between 100 μm and 1000 μm, between 100 μm and 500 μm, between 500 μm and 1000 μm, between 200 μm and 500 μm, or between 200 μm and 300 μm. In some embodiments, the width of the electrode line 204 is about 250 μm and the spacing between respective electrode lines 202 is about 250 μm. The electrode lines 204 can be made of silver (Ag) or other suitable conductive material. The one or more pairs of electrodes 202 in electronic contact with a respective gas sensing film of the gas sensing films 106-1, 106-2, and 106-3 are configured for measuring resistance changes across the gas sensing film upon chemical interaction of the gas sensing film with a target gas.

The interdigitated electrode layer 200 can be prepared by printing interdigitated electrode lines onto a surface of the substrate 102 or on a surface of the gas sensing film 106 using conductive printing ink. The electrodes can be printed via screen printing, gravure printing, flexographic printing, ink-jet printing, or other electrode printing methods known in the art. After the printing, the interdigitated electrode layer 200 is cured (see Example 1).

In some embodiments, the chemical sensor device 100 includes an inductive electrode 210, shown in FIG. 2B. Gas sensing film 106 is electronically connected to inductive coil 212. A microprocessor 214 detects changes in resistance and capacitance and converts these into an impedance change, due to the absorption of the analyte gas(es) by the gas sensing film. The impedance of the coil (the resistance and capacitance) changes as analyte gas is absorbed by gas sensing film 106.

### Sensor Cartridge

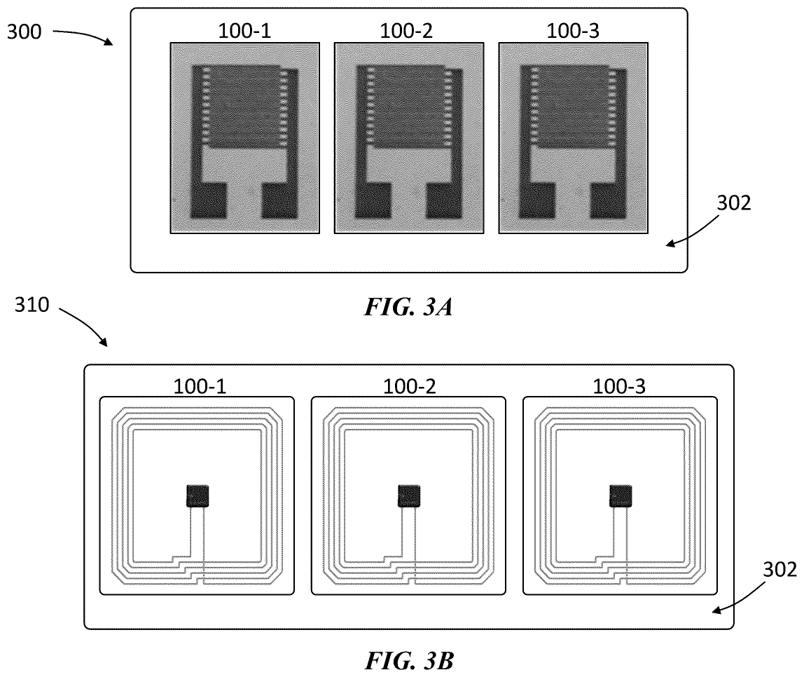

FIGS. 3A-3B are schematic illustrations of disposable gas sensing strips 300 and 310 in accordance with some embodiments. Sensing strip 300 illustrated in FIG. 3A consists of one or more chemical sensor devices 100-1, 100-2, and 100-3, each of which is configured according to the embodiment illustrated in FIG. 2A. Each device has a set of interdigitated electrodes with a gas sensing film either on top of the electrodes or between the electrodes and a substrate. The devices are mounted on a base 302 which is made of an insulating material. Sensing strip 310 illustrated in FIG. 3B consists of one or more chemical sensor devices 100-1, 100-2, and 100-3, each of which is configured according to the embodiment illustrated in FIG. 2B. Each device has an inductive coil with a gas sensing film either on top of the coil or between the coil and a substrate. The devices are mounted on a base 302, which is made of an insulating material.

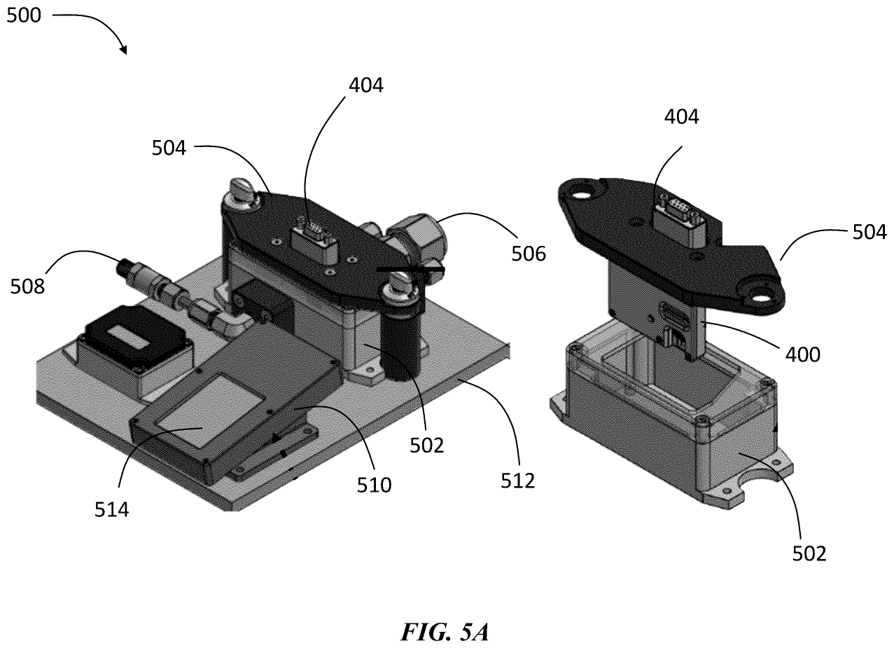

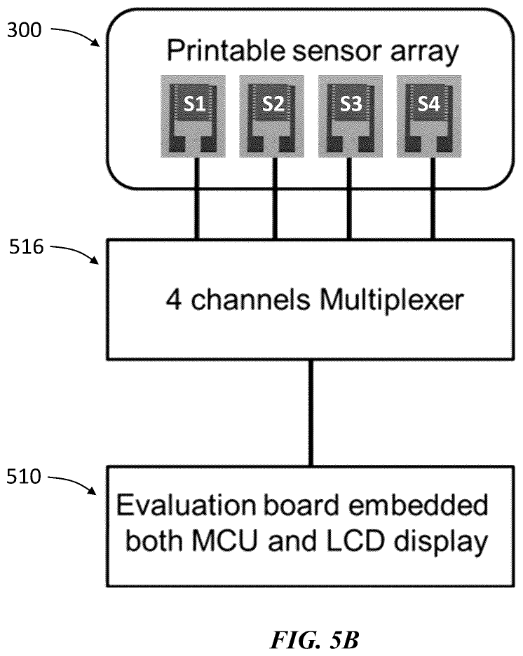

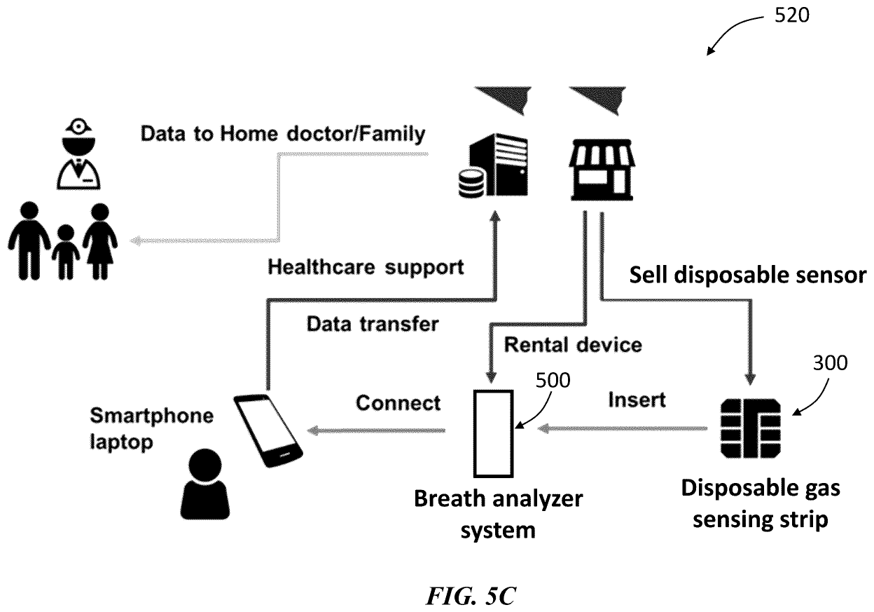

FIG. 4 is a schematic illustration of an exemplary prototype sensor cartridge 400. The sensor cartridge 400 is configured to receive the disposable gas sensing strip 300 and hold the sensing strip 300 during a measurement. The sensor cartridge 400 is configured to be inserted into a breath analyzer (e.g., a breath analyzer system 500 described with respect to FIG. 5A). In some embodiments, the sensor cartridge 400 as well as the sensing strip 300 are configured to be disposable (e.g., made of low-cost materials).

The sensor cartridge 400 includes a case 402 including a lower case half 406 and a top case half 414 which when assembled form the case 402 that is configured to surround the other components of the sensor cartridge 400. The lower case half 406 and the top case half 414 are mechanically coupled by a set of fasteners (e.g., screws 416) or clips. As used herein, “coupled” refers to being connected mechanically or electronically.

The sensor cartridge 400 also includes an electronic sensing circuit 408, a printed circuit board (PCB) 410, a connector 404, a cartridge (e.g., a slider) 412 for receiving and holding the connector 404 that are, when assembled, positioned inside the case 402. The sensing circuit 408 is electronically connected to the PCB 410. The sensing circuit 408 converts the analog signal, the resistance and/or capacitance changes of each of the gas sensing devices in sensing strip 300, into digital information that can be stored or displayed.

The connector 404 is configured to electronically couple to the sensing strip 300 so that the connector 404 and the sensing strip 300 are electronically coupled to the sensor cartridge 400. The connector 404 is configured to electronically connect the sensing electrodes 104 of the sensing strip 300 with the PCB 410 and sensing circuit 408 for the detection of resistance and/or capacitance changes in the gas sensing films 106-1, 106-2, and 106-3 of the sensing strip 300 upon chemical interaction with a target gas. For example, the connector 404 includes a strip socket that engages and mounts to the electrodes 104 of the sensing strip 300. The strip socket includes pins configured to electronically connect the sensor electrodes 104 with the PCB 410 when the sensing strip 300 is mounted in the socket (e.g., inserted inside the sensor cartridge 400).

### Breath Analyzer

FIG. 5A is a schematic illustration of a breath analyzer system 500 in accordance with some embodiments. The breath analyzer system 500 is for detecting one or more gases of NH₃, TMA, and H₂S. The breath analyzer system 500 includes the sensor cartridge 400 that is configured to hold the disposable gas sensing strip 300 described with respect to FIG. 3, a testing chamber 502, and a sensor cartridge holder 504. In some embodiments, the breath analyzer system 500 can be a portable device or a handheld device.

As shown in FIG. 5A, the sensor cartridge 400 can be connected or disconnected from the sensor cartridge holder 504 so that the connector 404 is accessible from the top portion of the sensor cartridge holder 504. The sensor cartridge holder 504 can be placed in contact with the testing chamber 502 by placing the sensor cartridge holder 504 on top of the testing chamber 502. For example, the sensor cartridge holder 504 can operate as a lid for the testing chamber 502. The sensor cartridge holder 504 is configured to be mechanically coupled with the testing chamber 502, so that the testing chamber 502 receives the sensor cartridge 400 with the sensing strip 300 inside the testing chamber 502 when the sensor cartridge holder 504 is mechanically coupled with the testing chamber 502.

As also shown in FIG. 5A, the testing chamber 502 is mechanically coupled with a board 512. The board 512 is configured to provide structural support for the breath analyzer system 500. The testing chamber 502 is further mechanically coupled with a gas inlet 506 and a gas outlet 508. The gas inlet 506 and the gas outlet 508 can be positioned at opposing sides of the testing chamber 502 so that a gas sample (e.g., breath) can enter the testing chamber 502 through the gas inlet 506 and exit the testing chamber 502 through the gas outlet 508. The sensor cartridge 400 is placed in the center of the testing chamber 502 directly opposite to the gas inlet 506. After sealing the testing chamber 502 by closing the top of the device (e.g., sensor cartridge holder 504), gas samples are injected through the gas inlet. In some embodiments, the gas inlet 506 includes, or is mechanically coupled with, a mouthpiece. A user can blow a breath sample through the mouthpiece so that the breath sample flows through the testing chamber 502 to the gas outlet 508.

The board 512 can be additionally electronically coupled with display 514 in encasement 510 which may house a microcontroller unit (MCU) with memory storage. The MCU can be configured to process and store information about detection events. The display 514 can be configured to output (e.g., display) information including detection results to a user. For example, the MCU with memory storage can be used to store values based on the resistance and/or capacitance change during a gas measurement and display a specific target gas and its concentration based on a pattern recognition algorithm and compute the concentration of the target gas.

The breath analyzer system 500 is configured to dynamically collect breath analytes (e.g., human breath) by timed sampling. Upon interaction with one or more target gases, the sensing strip 300 causes a response signal (e.g., a change in resistance and/or capacitance) to be dynamically recorded and processed to determine the presence and/or concentration of the one or more target gases by analysis of sensor array response pattern and magnitude of dynamic responses.

FIG. 5B is a schematic illustration of an exemplary electronic connection port 516 of sensor cartridge 400 in accordance with some embodiments. The port 516 is configured to form an electronic communication path between the disposable gas sensing strip 300 and the evaluation board 510 of the breath analyzer system 500. In FIG. 5B, the sensing strip 300 includes electronic connections for four gas sensing films (e.g., electronic connections S1, S2, S3, and S4 in FIG. 5B).

Data for signal analysis and processing are transferred from the sensing strip 300 to the evaluation board 510 via the electronic connection port 516. In FIG. 5B, the electronic connection port 516 includes a 4-channel multiplexer. Based on the sensor array response pattern and magnitude of sensing response, the display 514 can output specific target analyte identifications (e.g., NH₃, TMA, or H₂S) and their respective concentrations during breath analysis.

The breath analyzer system 500 is configured to distinguish different target gases based on the sensor array response pattern. The breath analyzer system 500 is further configured to compute the concentration of respective target gases based on sensor resistance change, and store and display information related to the measurement (e.g., identification and concentration of the target gas).

Pattern recognition of gases using gas sensor arrays has been described, e.g., by E. Kim et al. in “Pattern Recognition for Selective Odor Detection with Gas Sensor Arrays,” Sensors 2012 Nov. 23; 12(12):16262-73. DOI: 10.3390/s121216262. Breath analyzers have been described by Rigas et al in International Patent Publication No. WO 2014/063169, International Patent Publication No. WO 2020/123565 and in International Patent Publication No. WO 2021/021299; an electrochemical method and an electrochemical sensor for breath analysis have been described by Dincer et al in International Patent Publication No. WO 2020/234338; an electrochemical sensor has been described by Metters et al in International Patent Publication WO 2016/124874; a system for sensing and measuring ammonia in breath samples has been described by Killard et al in US Patent Publication U.S. Pat. No. 9,435,788; and gas sensors with inductive coils have been described by R. Potyrailo and C. Surman in “A Passive Radio-Frequency Identification (RFID) Gas Sensor With Self-Correction Against Fluctuations of Ambient Temperature”, Sens Actuators B: Chem. 2013, 185:587-593. All of the references cited herein are incorporated by reference in their entirety.

FIG. 5C is a schematic illustration of an exemplary environment 520 where the breath analyzer system 500 can operate in accordance with some embodiments. The breath analyzer system 500 is an economically viable detection system that can be distributed to general practitioners for autonomous, portable, and easy measurements utilizing disposable chemical sensor devices (e.g., the breath analyzer 500 utilizing the sensing strip 300). A medical device manufacturer or a distributor can provide the breath analyzer system 500 for rent or sale to users (e.g., patients, nurses, etc.). The medical device manufacturer can also sell the disposable sensing strip 300 utilized within the breath analyzer system 500. The user can conveniently take measurements at a location convenient for them (e.g., home, work, or during travel). The breath analyzer system 500 can communicate (e.g., via wireless or wired communication) with a personal device (e.g., a laptop computer, tablet computer, or smartphone) of the user to report detection results. The personal device can further transfer the reported detection results to a healthcare provider or other data collection unit. The healthcare provider can communicate the results further to a physician, the user and/or the user's family or caretakers.

The following examples further illustrate the present invention. These examples are intended merely to be illustrative of the present invention.

### EXAMPLES

### Example 1: Detailed Sensing Material Composition for Ammonia (NH₃) Sensing and Printed Sensor Fabrication Process

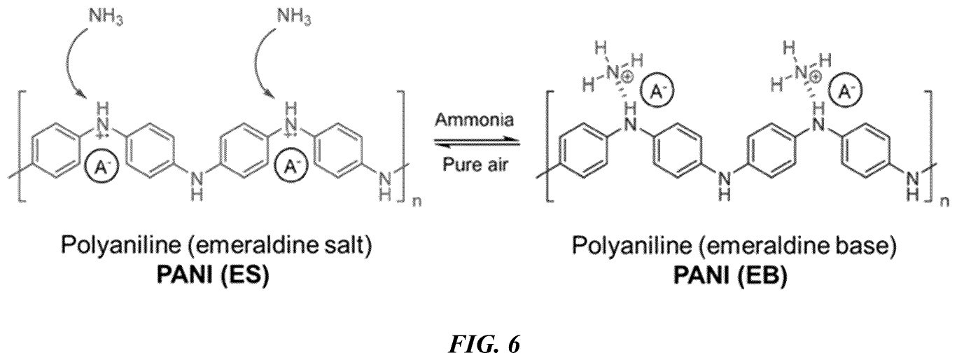

FIG. 6 illustrates a mechanism for PANI-based detection of NH₃.

As shown in FIG. 6, NH₃ deprotonates the amine group in the emeraldine salt(ES) form of PANI, converting it to the emeraldine base (EB) form with a corresponding drop in conductivity by several orders of magnitude. By proper selection of the dopant protonic acid, this pH sensitivity can be further adapted to target different gas analytes selectively.

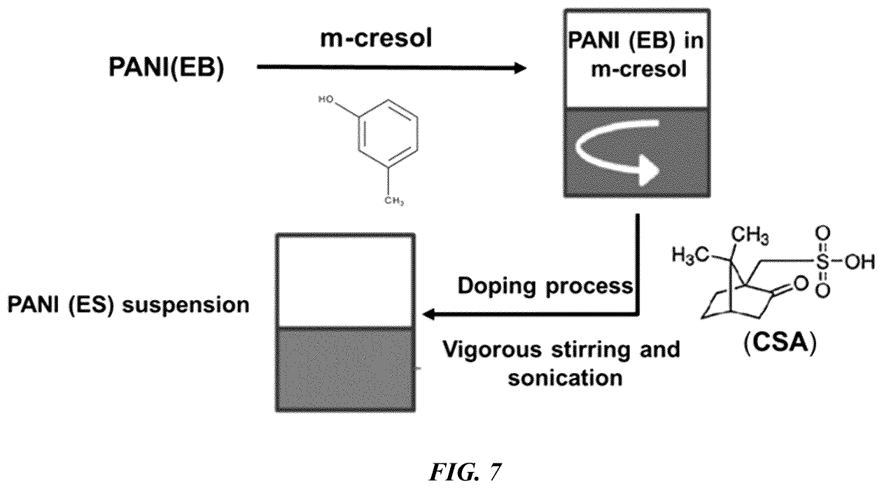

PANI-based conducting polymers doped with CSA were prepared by obtaining PANI (EB), camphor sulfonic acid (CSA), and m-cresol (PANI (EB), CSA, and m-cresol available, for example, from Sigma-Aldrich). FIG. 7 is a schematic illustration of one possible preparation route of emeraldine salt of PANI (PANI(ES)) doped with CSA. PANI (EB) was dissolved in m-cresol using an ultrasonic bath for 4 hours at 50° C. to form PANI (EB) solution. After forming homogeneous solution, camphor-10-sulfonic acid (CSA) was added into the PANI (EB) solution in a molar ratio of CSA:PANI=1.5 (PANI_CSA_{1.5}) and treated in an ultrasonic bath with vigorous stirring for another 4 hours at 50° C. The final concentration of PANI_CSA_{1.5} solution was around 2.5% (w/w).

In an alternative procedure, PANI (EB) was mixed with CSA in a molar ratio of CSA:PANI=1.5 (PANI_CSA_{1.5}), using an agate mortar and pestle in an ambient atmosphere. An appropriate quantity of the resulting mixture was combined with m-cresol, and treated in an ultrasonic bath for 8 hours at 50° C. to form 2.5% (w/w) of PANI_CSA_{1.5} solution.

To prepare a stock solution for the dip coating process, dichloromethane was used to further dilute PANI_CSA_{1.5} m-cresol solution to form 0.2% (w/w) solution. The total volume of solution was 1000 ml.

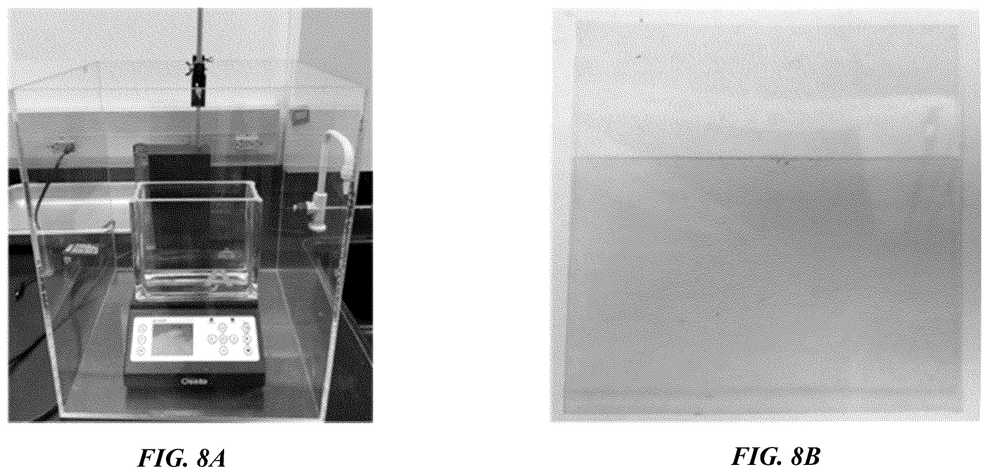

The dip coating process was carried out using an Ossila dip coater (Ossila Ltd, Sheffield, UK, FIG. 8A). A bare PET substrate (5.5×5.5 inch) was fixed on the dip coater with a clamp, immersed into the PANI_CSA_{1.5} solution, and withdrawn at a speed of 15 mm/s. FIG. 8A illustrates a bare PET substrate (5.5×5.5 inch) with PANI_CSA_{1.5} thin films prepared using the Ossila dip coater. FIG. 8B illustrates a PANI_CSA_{1.5} thin film dip coated onto the bare PET substrate.

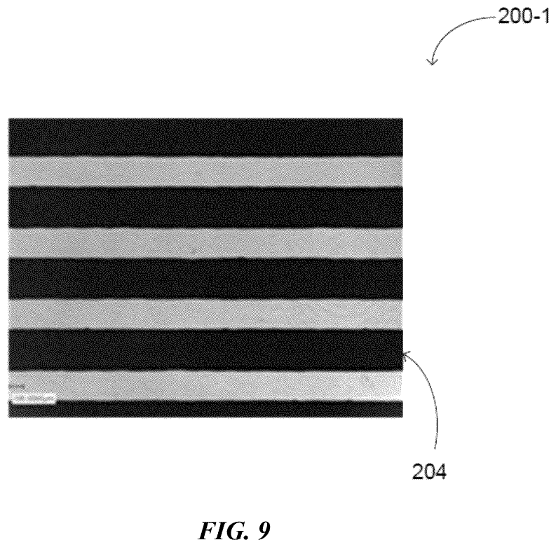

The PANI_CSA_{1.5} coated PET substrate illustrated in FIG. 8B was placed in an oven at 80° C. for 16 hours to remove solvent completely before electrode printing. A custom-designed printing screen including 24 paired electrode patterns with 250 μm spacing (e.g., available from Sefar AG, Switzerland) was used to prepare an interdigitated electrode layer (e.g., as shown in FIG. 2A). The screen printing was performed with a DEK Neo Horizon 03 iX printing machine. Henkel ECI 1010 silver screen printing ink was used to print the electrodes. FIG. 9 illustrates an optical microscope image of a portion 200-1 of the prepared interdigitated electrode layer 200 described with respect to FIG. 2A. Each individual interdigitated electrode (IDE) contained twelve pairs of electrodes. The width of the electrode line is 250 μm and the spacing between lines is about 250 μm.

Eighteen individual dip coated PANI_CSA_{1.5} sensors were fabricated on a single sheet. The average sensing film thickness was around 30 nm. The measured sensor resistances ranged from 1.5 kΩ to 3 kΩ. The individual sensor strips were cut out to insert into the sensor holders for gas testing measurement.

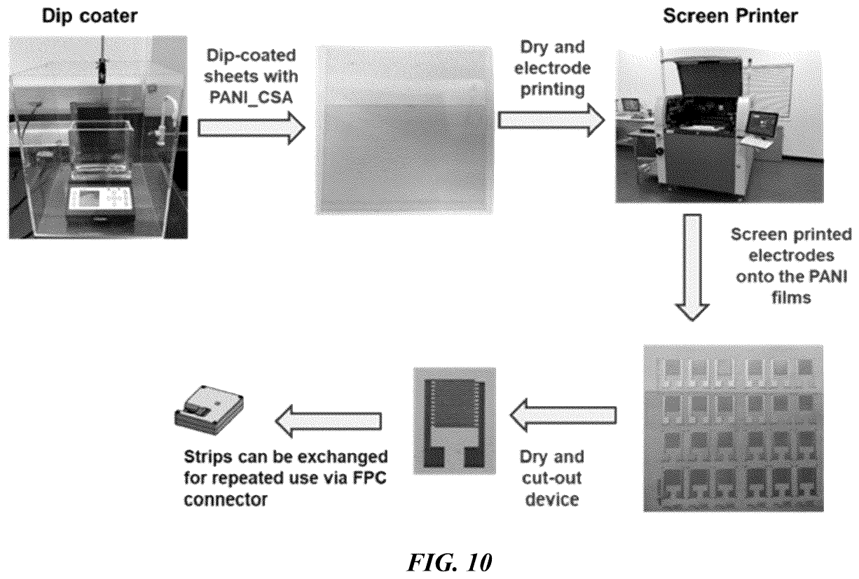

FIG. 10 illustrates the fabrication process of chemical sensors incorporating PANI-based conducting polymers doped with CSA in accordance with some embodiments. The fabrication process of FIG. 10 is for fabricating chemical sensors where the PANI_CSA_{1.5} thin films are positioned between the substrate and the interdigitated electrodes. The fabrication process included dip coating PET substrates in the PANI_CSA_{1.5} solution to form PANI_CSA_{1.5} thin films. The PANI_CSA_{1.5} thin films were then dried in an oven. After drying, the interdigitated electrodes were printed onto the PANI_CSA_{1.5} thin films using a screen printer. The printed interdigitated electrodes were then dried and individual sensor strips were cut out. The individual sensor strips were suitable for inserting into the sensor cartridge 400 described with respect to FIG. 4.

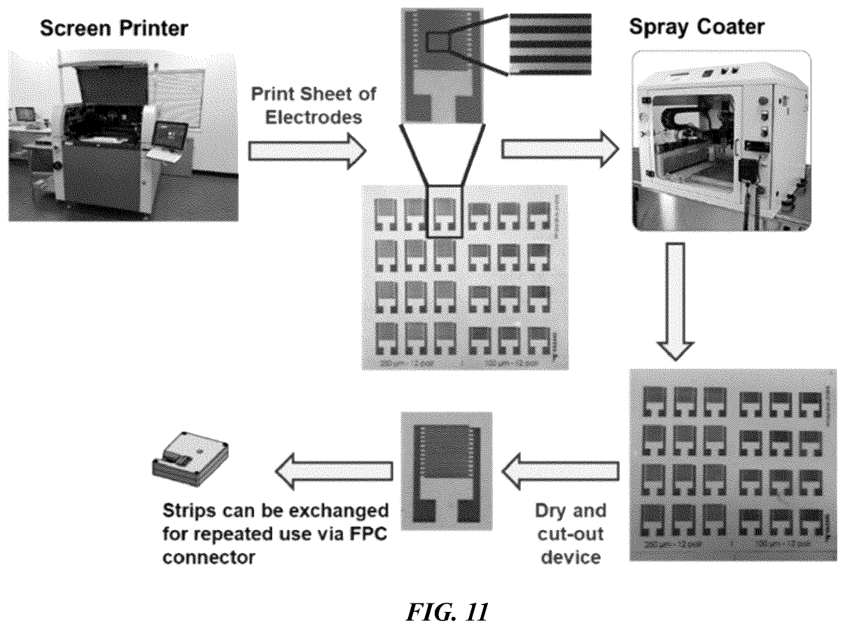

FIG. 11 illustrates the fabrication process of PANI-based conducting polymers doped with CSA in accordance with some embodiments. The fabrication process of FIG. 11 is for fabricating chemical sensors where the interdigitated electrodes are positioned between the substrate and the PANI_CSA_{1.5} thin films. 24 electrodes with 250 μm spacing were printed onto the bare PET substrate. After electrode fabrication, PANI_CSA_{1.5} solution was spray coated onto the electrodes using a multi-axis spraying system (ExactaCoat, Sono-Tek Corporation). The concentration of PANI_CSA_{1.5} spraying solution was 0.02% (w/w). The substrate sheet was kept at 25° C. After spray coating, the whole sheet of PANI_CSA_{1.5} sensors was placed in an oven at 80° C. for 16 hours to completely remove solvents. The average sensing film thickness was about 30 nm. The sensor resistances ranged from 1.5 kΩ to 3 kΩ. 24 individual spray coated PANI_CSA_{1.5} sensors were fabricated on a single sheet. Individual sensor strips are cut out to insert into the sensor holders for gas testing measurement. The individual sensor strips were suitable for inserting into the sensor cartridge 400 described with respect to FIG. 4.

### Example 2: Sensing Material Composition for Trimethylamine (TMA) Sensing and Printed Sensor Fabrication Process

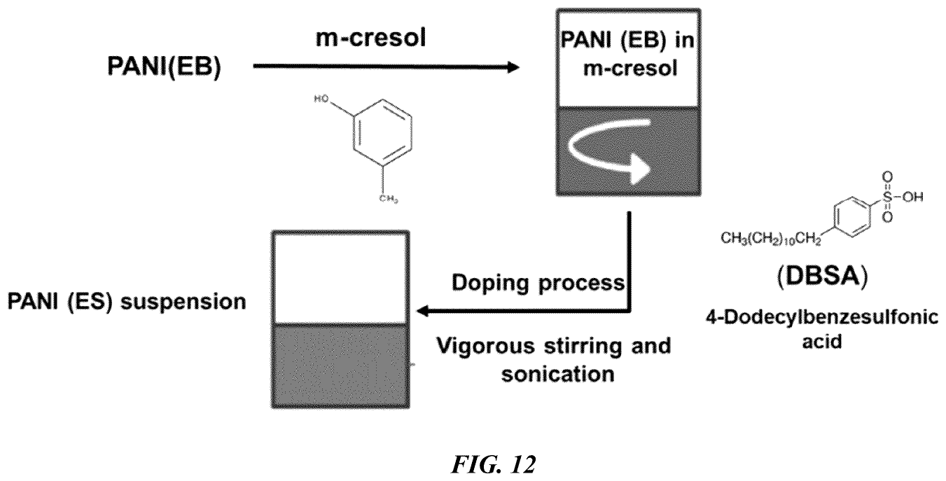

PANI-based conducting polymers doped with DBSA were prepared by obtaining PANI (EB), DBSA, m-cresol, and dichloromethane (Sigma-Aldrich). FIG. 12 is a schematic illustration of one possible preparation route of emeraldine salt of PANI (PANI(ES)) doped with DBSA. PANI (EB) was dissolved in m-cresol using an ultrasonic bath for 4 hours at 50° C. to form PANI (EB) solution. After forming homogeneous solution, DBSA was added into the PANI (EB) solution in a molar ratio of DBSA:PANI=1.5 (PANI_DBSA_{1.5}) and treated in an ultrasonic bath with vigorous stirring for another 4 hours at 50° C. The final concentration of PANI_DBSA_{1.5} solution was around 2.5% (w/w).

In an alternative procedure, PANI (EB) was mixed with DBSA in a molar ratio of DBSA:PANI=1.5 (PANI_DBSA_{1.5}). An appropriate quantity of the resulting mixture was placed into m-cresol, and treated in an ultrasonic bath for 8 hours at 50° C. to form 2.5% w/w PANI_DBSA_{1.5} solution.

As described in Example 1 for PANI_CSA_{1.5}, both dip coating (coating/printing process, FIG. 10) and spray coating (printing/coating process, FIG. 11) were used to fabricate PANI_DBSA_{1.5} sensors for TMA sensing. Compared to PANI_CSA_{1.5} sensing films, PANI_DBSA_{1.5} thin films were less conductive. To meet the operational sensor resistance range (1 to 3 kΩ) of the chemical sensor device, the thicknesses of PANI_DBSA_{1.5} sensors were controlled to be about 50 nm.

The sensing mechanism of TMA is similar to that of NH₃, as described in Example 1 and shown in FIG. 6, Similarly as for NH₃, TMA deprotonates the amine group in the emeraldine salt(ES) form of PANI, converting it to the emeraldine base (EB) form with a corresponding drop in conductivity.

### Example 3: Sensing Material Composition for Hydrogen Sulfide Sensing and Printed Sensor Fabrication Process

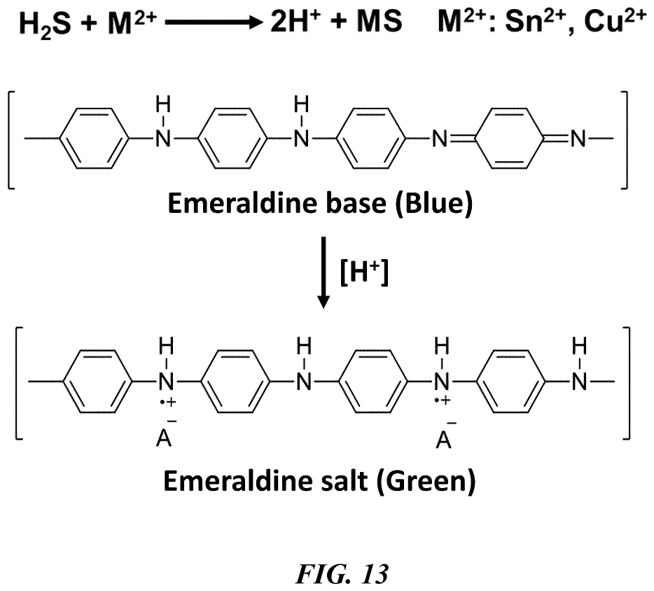

FIG. 13 illustrates a sensing mechanism of Sn/Cu (II) doped PANI composites for H₂S. Sn/Cu (II) doped polyaniline (PANI) composites demonstrated great potential for H₂S sensing. The change in resistance of the Sn/Cu (II) doped PANI film upon exposure to H₂S can be explained by the formation of a metal sulfide and release of two H⁺ from the H₂S molecules, in accordance with the chemical reaction shown in the Figure. The H⁺ protonates the emeraldine PANI and leads to increased conductivity.

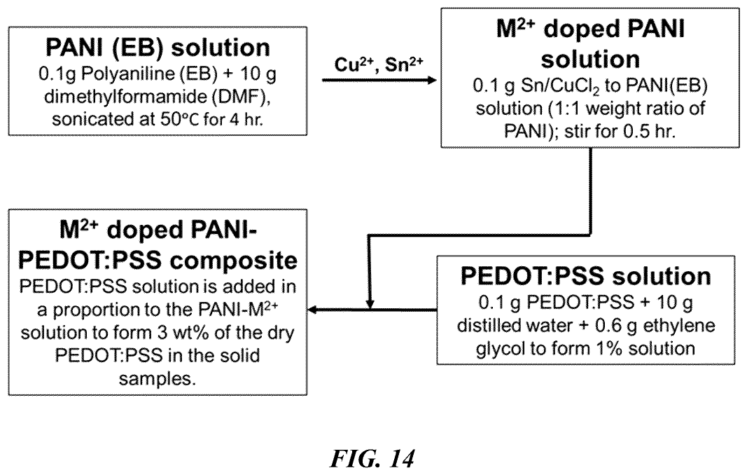

A hybrid material was prepared for sensing H₂S, based on PANI, Sn(II) chloride, and PEDOT:PSS composite. Since the initial resistivity of metal salt-doped PANI is high in the native state, PEDOT:PSS, which is known for its high conductivity of up to 500 S/cm, was added to facilitate charge transport in the material and to match the electronic properties of the hybrid material with the resistance range required for sensing. FIG. 14 is a schematic illustration of formulation routes for Sn/Cu (II) doped PANI-PEDOT:PSS composite solutions for spray coating or dip coating.

Similar to Example 1 and Example 2, both dip coating (coating/printing process, FIG. 10) and spray coating (printing/coating process, FIG. 11) were used to fabricate M²⁺ doped PANI-PEDOT:PSS composite for H₂S sensing. Due to decreasing resistance of sensors after H₂S exposure, the thicknesses of PANI-PEDOT:PSS composite sensors were controlled to be about 100 nm, and sensor resistance was between 6 and 8 kΩ.

### Example 4: Sensing Performance Toward NH₃ Under 90% RH (Simulated Human Breath Condition)

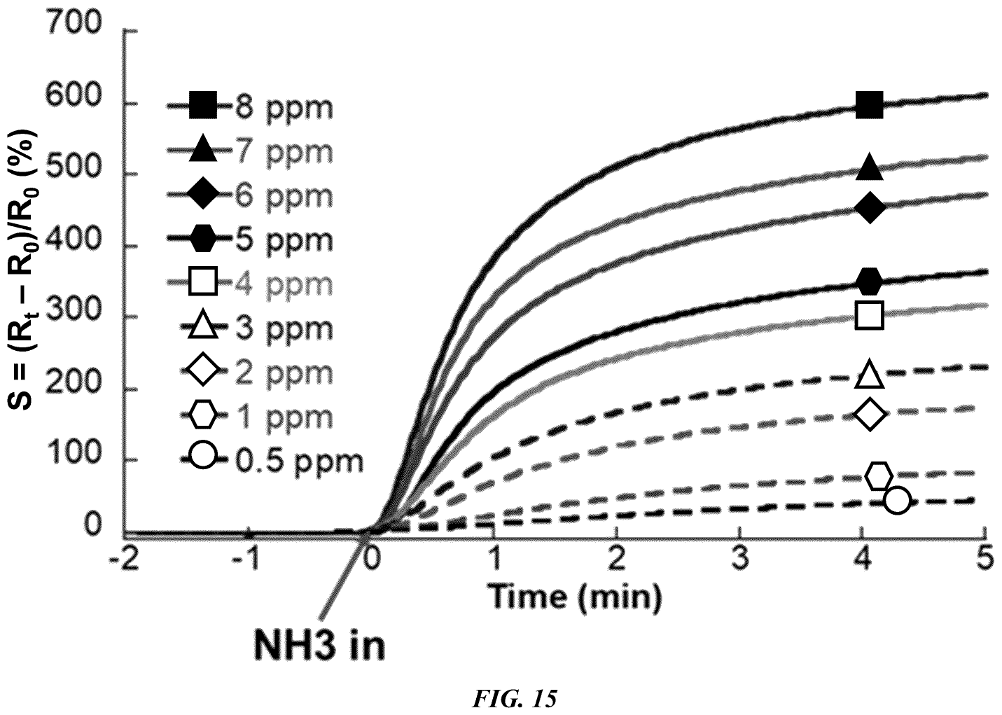

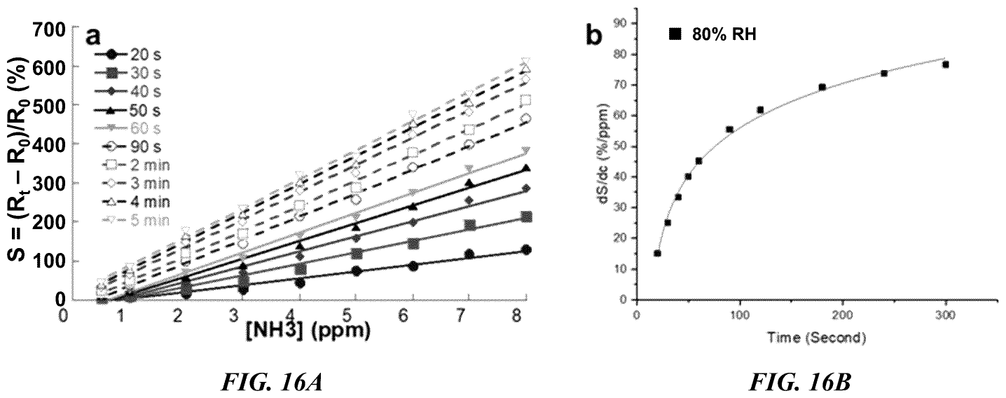

To study NH₃ sensing performance of PANI_CSA_{1.5} under 90% RH condition (the approximate RH of human breath), the changes of PANI_CSA_{1.5} resistance after 5-minute NH₃ exposure were measured and sensor response(S) was defined as the relative resistance change, S=(Rₜ−R₀)/R₀*100%, where Rt equals the resistance of the sensor after a specific time of exposure to NH₃, and R₀ equals sensor resistance at ambient condition before NH₃ exposure. FIGS. 15, 16A, and 16B are graphical illustrations of resistance changes of PANI_CSA_{1.5} composites when exposed to NH₃ gas. The sensitivity of PANI_CSA_{1.5} toward 0.5 to 8 ppm NH₃-90% RH mixture for 5-minute exposure is demonstrated. As shown in FIG. 15, PANI_CSA_{1.5} sensing response followed a log-type behavior vs NH₃ exposure time, a typical behavior for a gas diffusion process. The sensor response(S) increased with elevated NH₃ concentration. The calibration curves of NH₃ concentration vs. sensor response(S) at a specific testing time (i.e., 20, 30, 40, 50, 60, 90, 120, 180, 240, and 300 seconds) are shown in FIG. 16A. The calibration curves show a linear relationship between sensor response and concentration of NH₃. The slopes of the calibration curves (dS/dc, S: sensor response, c: concentration of NH₃) also followed a log-type behavior with NH₃ exposure time. (FIG. 16B). A transfer function c (NH₃)=f (S, time) was created with fitting parameters under a specific humidity level. The fitting equation could be simplified as S=c*A1*In (A2*t+1), where A1 and A2 depend on the humidity level.

### Example 5: Sensing Performance Toward TMA Under 90% RH (Simulated Human Breath Condition)

The sensing performance of PANI_DBSA_{1.5} sensors toward 0.1 to 1 ppm TMA-90% RH mixtures was demonstrated. Four PANI_DBSA_{1.5} sensors were tested toward 0.1, 0.2, 0.5, and 1 ppm TMA under 90% RH condition. The resistance changes of PANI_DBSA_{1.5} sensors were measured for 4-minute TMA exposure and sensor response(S) was defined as the relative resistance change, S=(Rₜ−R₀)/R₀*100%, where Rₜ is the resistance of the sensor after exposure to the analyte gas for a specific time, and R₀ is sensor resistance at ambient condition before gas exposure.

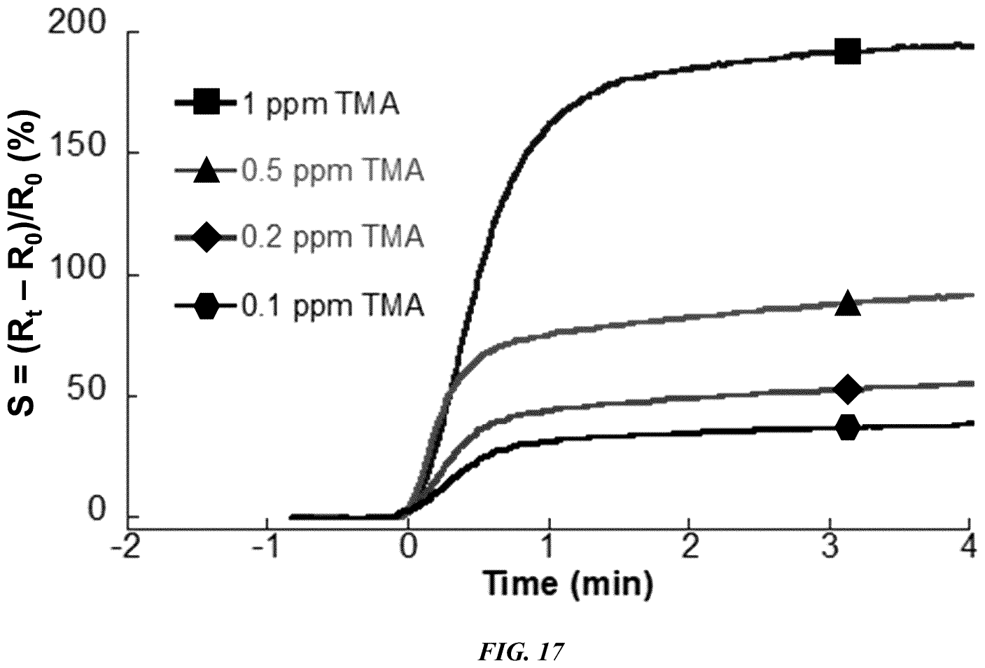

FIG. 17 is a graphical illustration of resistance changes of PANI_DBSA_{1.5} sensors when exposed to TMA gas. Similar to NH₃ responses, dynamic TMA sensing responses of PANI_DBSA_{1.5} sensors followed a log-type behavior vs. TMA exposure time, as shown in FIG. 17. Sensing response increased with elevated TMA concentration. The detection limit of TMA under 90% RH condition was measured to be as low as 0.1 ppm.

### Example 6: Sensing Performance Toward H₂S Under 90% RH (Simulated Human Breath Condition)

The performance of Sn(II) doped PANI-PEDOT:PSS hybrid material toward 1 to 4 ppm H₂S under 90% relative humidity (RH) conditions was demonstrated. Four sensors were used to test toward 1, 2, 3, and 4 ppm H₂S-90% RH mixtures. The resistance changes of each sensor were measured for 10-minute H₂S exposure and sensor response(S) was defined similarly as in Examples 4 and 5.

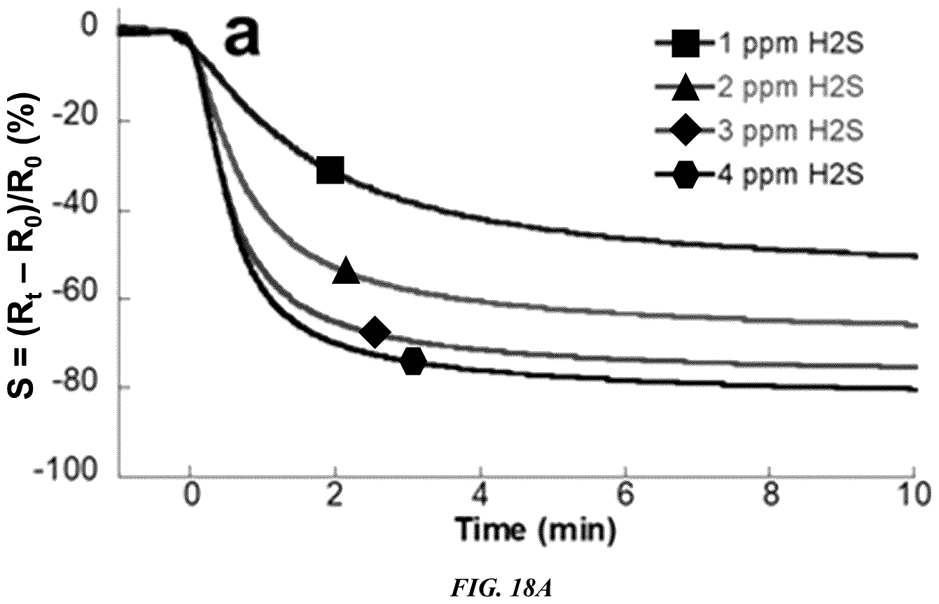

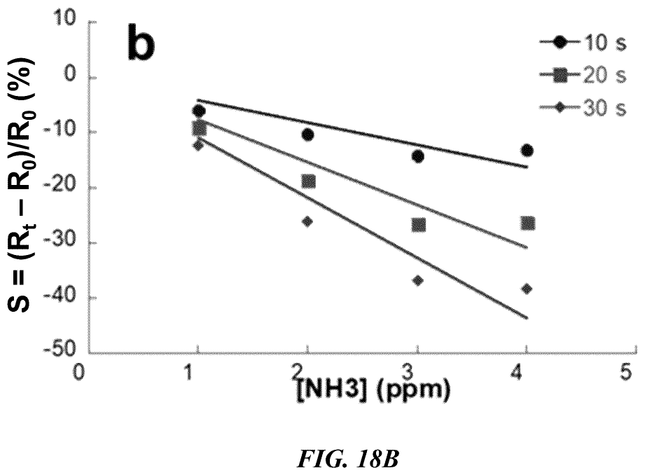

FIGS. 18A and 18B are graphical illustrations of resistance changes of Sn(II) doped PANI-PEDOT:PSS hybrid material when exposed to H₂S under 90% RH. FIG. 18A shows dynamic responses of Sn(II) doped PANI-PEDOT:PSS sensors toward 1 to 4 ppm H₂S under 90% RH conditions. Unlike positive resistance changes for NH₃ and TMA sensing responses, a decrease in resistance of the Sn(II) doped PANI sensors occurred upon exposure to H₂S. As shown in FIG. 13, H₂S is known to react with metal ions in a +2 oxidation state, such as Sn(II) or Cu (II), to form a metal sulfide (MS) and release H⁺, which further dopes the PANI and decreases the resistance of the polymer.

Calibration curves of Sn(II) doped PANI-PEDOT:PSS sensors toward 1-4 ppm H₂S with 10 to 30 seconds exposure time are depicted in FIG. 18B. A linear relationship appears between the H₂S sensing responses and the concentration of H₂S. A linear calibration curve can be used to quantify sub-ppm H₂S concentration under 90% RH with less than 30 seconds exposure time.

### Example 7: Three-Element Sensor Array for Differentiating H₂S, TMA, NH₃, and TMA-NH₃ Mixtures Under 90% RH

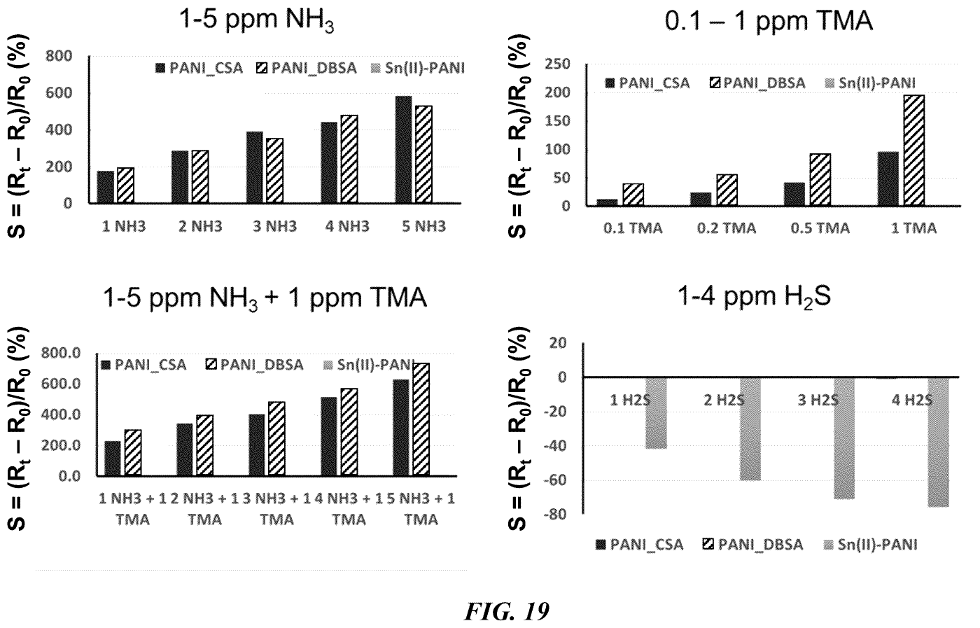

Here, an array of three film sensors consisting of Sn(II) doped PANI-PEDOT:PSS, PANI_CSA_{1.5}, and PANI_DBSA_{1. 5} sensors was used to determine the response toward 1 to 4 ppm H₂S, 0.1 to 1 ppm TMA, 1-5 ppm NH₃, and 1-5 ppm NH₃-1 ppm TMA mixtures, under 90% RH. FIG. 19 shows the sensor responses of the individual sensors in the sensor array toward these four analytes.

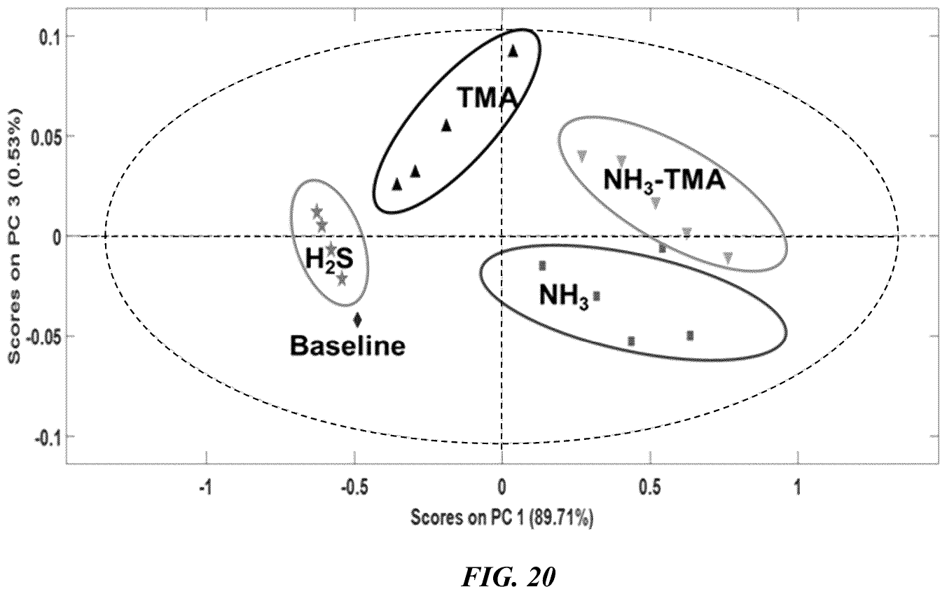

For further data analysis, a classification algorithm was used to classify the responses of the sensor array to the four different analytes. Here, the principal component analysis (PCA) technique was used to demonstrate a classification of H₂S, TMA, NH₃, and TMA-NH₃ mixtures based on sensor array response patterns. FIG. 20 is a graphical illustration of the classification of H₂S, TMA, NH₃, and TMA-NH₃ based on the PCA. Differences in the clustering of each analyte indicated the clear separation of PCA grouping of 1-4 ppm H₂S, 1-5 ppm NH_{3, 0.1} to 1 ppm TMA, and 1-5 ppm NH₃-1 ppm TMA, which demonstrated the effective discrimination of these 4 analytes.

Besides forming a simple three-film sensor array based on the sensor resistance change, three different PANI-based sensing materials were applied to a sensor array based on LCR (inductor-capacitor-resistor) transducers to selectively detect H₂S, TMA, and NH₃ wirelessly.

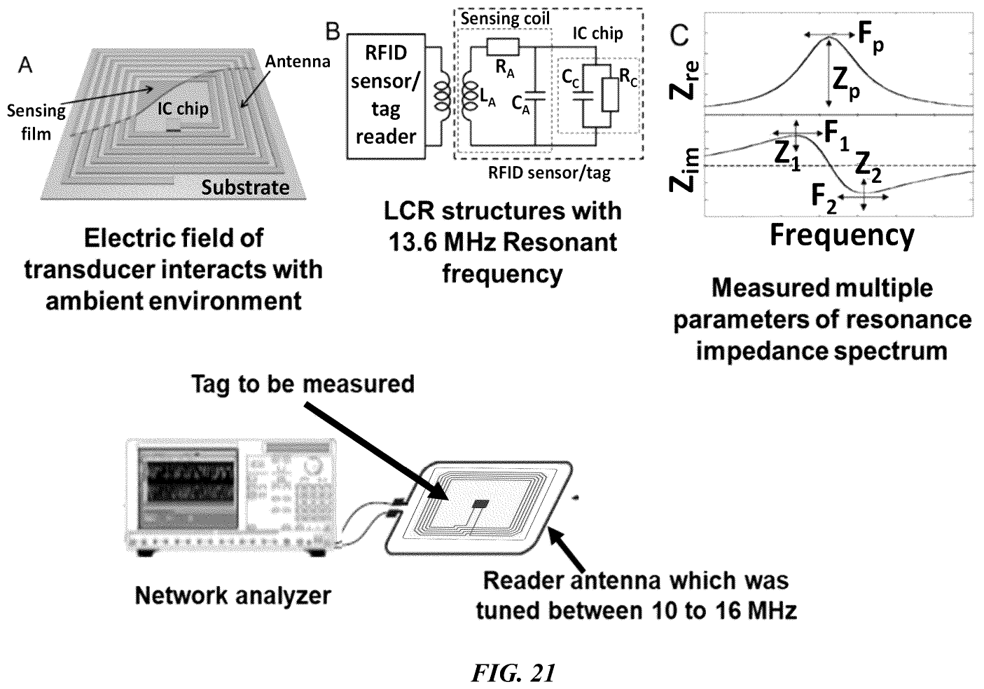

FIG. 21 is a schematic illustration of a gas sensor in which the sensing material was coated onto an inductive coil described earlier and depicted in FIGS. 2B and 3B. An external pickup coil was used to read the resonance frequency and intensity of the impedance peak of the sensing tag. When the inductive coil was exposed to the target gas, the sensing materials not only changed resistance which affected the intensity of the impedance spectrum, but also changed the capacitance for resonance frequency shift. Compared to only measuring the change of resistance, the inductive sensing element was able to measure both resistance and capacitance simultaneously.

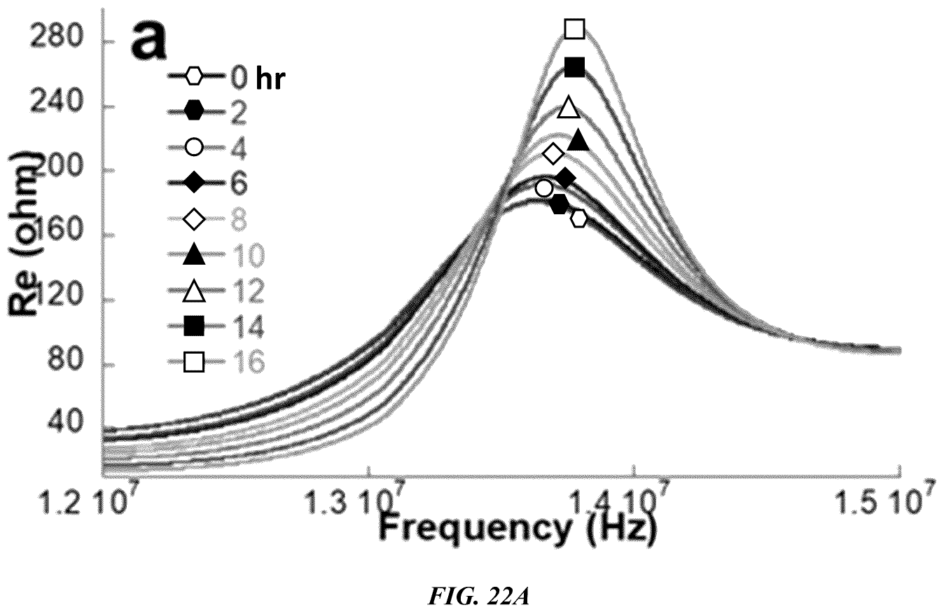

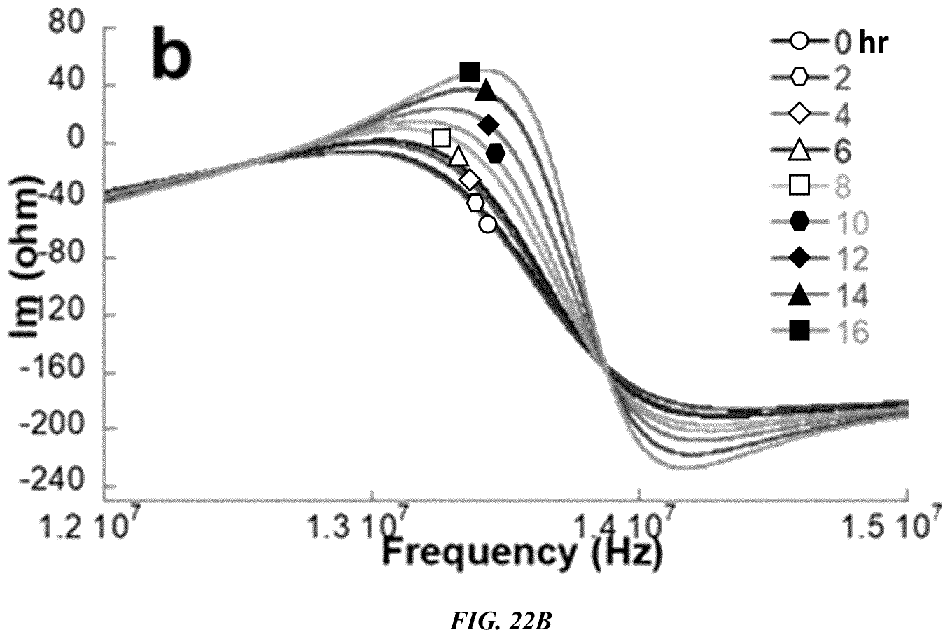

FIGS. 22A and 22B are graphical illustrations of resonance impedance spectrum changes of PANI_CSA_{0.5} coated inductive coils for TMA-NH₃ concentration monitoring. FIG. 22A illustrates the real part of the impedance spectrum and FIG. 22B illustrates the imaginary part of the impedance spectrum. It can be easily understood that a sensor array comprising separate coils coated with Sn(II) doped PANI-PEDOT:PSS, PANI_CSA_{1.5}, and PANI_DBSA_{1. 5} will also easily distinguish and resolve NH₃, TMA, and H₂S analyte gases similar to the sensor array described in the previous Example 7. Here changes in resonance frequency and resonance amplitude rather than resistance changes are utilized.

### Remarks

The above description and drawings are illustrative and are not to be construed as limiting. Numerous specific details are described to provide a thorough understanding of the disclosure. However, in certain instances, well-known details are not described in order to avoid obscuring the description. Further, various modifications may be made without deviating from the scope of the embodiments.

Unless stated otherwise, generally, the term “about” is meant to encompass a variance or range of ±10%.

We claim:

1. A system for differentially detecting ammonia (NH₃), trimethylamine (TMA), and a combination of NH₃ and TMA, comprising:
a sensor device;
a microcontroller unit (MCU); and
a memory comprising instructions, which when executed by the MCU, use principal component analysis (PCA) technique for analyzing sensor array response patterns measured by the sensor device,
wherein the sensor device comprises:
an interdigitated electrode layer including multiple pairs of electrodes;
a gas sensor array including multiple gas sensing films electronically coupled with the interdigitated electrode layer, a respective gas sensing film of the multiple gas sensing films configured to chemically interact with a target gas, wherein:
one or more pairs of electrodes of the interdigitated electrode layer are mechanically coupled to the respective gas sensing film of the gas sensor array,
the one or more pairs of electrodes are configured to concurrently measure resistance changes across the respective gas sensing film upon chemical interaction with the target gas, and
the multiple gas sensing films include:
polyaniline (PANI)-based conducting polymer doped with camphor sulfonic acid (CSA) for chemically interacting with NH₃ and TMA; and
PANI-based conducting polymer doped with 4-dodecylbenzenesulfonic acid (DBSA) for chemically interacting with TMA and NH₃; and
wherein the system is configured to differentially detect (i) NH₃, (ii) TMA, and (iii) a combination of NH₃ and TMA, by applying the PCA technique to the resistance changes measured with the multiple gas sensing films.

2. The system of claim 1, wherein:
the multiple gas sensing films further comprises metal salt doped PANI with poly(3,4-ethylenedioxythiophene) polystyrene sulfonate (PEDOT:PSS) composite for chemically interacting with H₂S, and
the system is configured to further differentially detect H₂S by applying the PCA technique to the resistance changes measured with the multiple gas sensing films.

3. The system of claim 2, which is configured to have a detection limit of 0.1 ppm to 0.5 ppm for at least one of NH₃, TMA, and H₂S.

4. The system of claim 2, wherein:
a molar ratio of CSA to PANI is in a range of 1 to 1.5;
a molar ratio of DBSA to PANI is in a range of 1 to 1.5; and
a weight percentage of PEDOT:PSS in metal salt doped PANI with PEDOT:PSS composite is in a range of 1 wt % to 4 wt %.

5. The system of claim 1, wherein a width of an electrode line in the interdigitated electrode layer is between 100 μm and 1000 μm and a spacing between respective electrodes in the electrode line is between 100 μm and 1000 μm.

6. The system of claim 1, wherein a thickness of the respective gas sensing film is less or equal to 100 nm.

7. The system of claim 1, wherein the multiple pairs of electrodes are made of Ag.

8. The system of claim 1, wherein the multiple gas sensing films in the gas sensor array are positioned adjacent to each other.

9. The system of claim 1, further comprising a substrate mechanically coupled with the interdigitated electrode layer, wherein the interdigitated electrode layer is positioned between the substrate and the gas sensor array.

10. The system of claim 1, further comprising a substrate mechanically coupled with the interdigitated electrode layer, wherein the gas sensor array is positioned between the substrate and the interdigitated electrode layer.

11. The system of claim 1, wherein:
the gas sensor array is positioned on a first surface of a substrate, and
the sensor device further includes an additional gas sensor array positioned on a second surface of the substrate, the second surface being opposite to, and parallel with, the first surface of the substrate.

12. The system of claim 1, further comprising:
a testing chamber coupled with a gas inlet and a gas outlet at opposing sides of the testing chamber; and
a sensor device holder configured to be coupled with the testing chamber so that the testing chamber receives the sensor device inside the testing chamber when the sensor holder device is coupled with the testing chamber.

13. A method for differentially detecting ammonia (NH₃), trimethylamine (TMA), and a combination of TMA and NH₃, the method comprising:
obtaining the sensor system of claim 1;
concurrently measuring, by the sensor system, resistance changes across a respective gas sensing film of multiple gas sensing films upon chemical interaction with a target gas; and
applying the PCA technique to the resistance changes measured across the respective gas sensing film of multiple gas sensing films upon chemical interaction with the target gas to differentially detect (i) NH₃, (ii) TMA, and (iii) the combination of NH₃ and TMA.

14. The method of claim 13, wherein:
a concentration of (i) NH₃ is 1 ppm to 5 ppm NH₃, a concentration of (ii) TMA is 0.1 ppm to 1 ppm TMA, and in (iii) the combination of NH₃ and TMA, a concentration of NH₃ is 1 ppm to 5 ppm and a concentration of TMA is 1 ppm.

15. The method of claim 13, wherein:
(i) NH₃, (ii) TMA, and (iii) the combination of NH₃ and TMA are detected from sample of breath having up to 90% relative humidity.

16. A method for differentially detecting ammonia (NH₃), trimethylamine (TMA), hydrogen sulfide (H₂S), and a combination of TMA and NH₃, the method comprising:
obtaining the sensor system of claim 2;
concurrently measuring, by the sensor system, resistance changes across a respective gas sensing film of multiple gas sensing films upon chemical interaction with a target gas; and
applying the PCA technique to the resistance changes measured across the respective gas sensing film of multiple gas sensing films upon chemical interaction with the target gas to differentially detect (i) NH₃, (ii) H₂S, (iii) TMA, and (iv) the combination of NH₃ and TMA.

17. The method of claim 16, wherein:
a concentration of (i) NH₃ is 1 ppm to 5 ppm NH₃, a concentration of (ii) H₂S is 1 ppm to 4 ppm, a concentration of (iii) TMA is 0.1 ppm to 1 ppm TMA, and in (iv) the combination of NH₃ and TMA, a concentration of NH₃ is 1 ppm to 5 ppm and a concentration of TMA is 1 ppm.

18. The method of claim 16, wherein:
(i) NH₃, (ii) H₂S, (iii) TMA, and (iv) the combination of NH₃ and TMA are detected from sample of breath having up to 90% relative humidity.
